(19) United States
(12) Patent Application Publication
(10) Pub. No.: US 2024/0003741 A1
(43) Pub. Date: Jan. 4, 2024

(54) A RAMAN PROBE AND APPARATUS AND METHOD FOR NON-INVASIVE IN VIVO MEASUREMENT OF ANALYTE PRESENCE OR CONCENTRATION

(71) Applicant: RSP SYSTEMS A/S, Odense S (DK)

(72) Inventors: Martin SCHJOLDAGER NIELSEN, Odense S (DK); Joshua John GIBSON, Cambridge (GB); Thomas James WATSON, Cambridge (GB)

(21) Appl. No.: 18/033,247

(22) PCT Filed: Oct. 22, 2021
(86) PCT No.: PCT/EP2021/079414

§ 371 Date: Apr. 21, 2023

(30) Foreign Application Priority Data
Oct. 23, 2020 (GB) 2016873.8

(51) Int. Cl. G01J 3/10; G01J 3/44; G01N 21/65; A61B 5/1455

(52) U.S. Cl. CPC G01J 3/10; G01J 3/44; G01N 21/65; A61B 5/1455; G01N 2201/06113; A61B 2562/0238; G01J 2003/102; G01J 2003/4424

(57) ABSTRACT

The present invention relates to an apparatus and method for non-invasive in vivo measurement, by Raman spectroscopy, of glucose present in interstitial fluid in the skin of a subject. The apparatus comprises at least one detector; a plurality of vertical-cavity surface-emitting lasers spatially distributed around the at least one detector, for irradiating the skin of a subject; wherein the at least one detector is configured to receive Raman scattered radiation transmitted from the sample in response to the received radiation from the vertical-cavity surface-emitting lasers.

The present invention relates to an apparatus and method for non-invasive in vivo measurement, by Raman spectroscopy, of glucose or other analyte present in a subject and typically in the skin of a subject. Typically, the apparatus and method is for measurement of glucose or other analyte present in the interstitial fluid in the skin of a subject. The invention also relates to a Raman probe for use in varied fields such as biochemistry, medicine, agriculture, pharmaceuticals, process control/Quality control, forensic applications and technologies, chemical production, material analysis and environmental monitoring.

The use of Raman spectroscopy for the transdermal in vivo measurement of glucose or other analyte present in skin is known. Our previous international applications WO 2018/103943 A1, WO 2016/034448 A1, and WO 2011/083111 A1 describe earlier iterations of such devices and provide details as to how they can function to determine the analyte level in the skin of a subject. Typically, the determination is made with respect to the analyte level in interstitial fluid within the skin.

The devices and methods work well and provide means for non-invasive measurement of, for example, the glucose level within a user's interstitial fluid, which correlates with the user's blood glucose level.

In general, a sample is irradiated by monochromatic light, such as light from a laser. The sample scatters the monochromatic light back to a detector which then analyses its spectrum. Usually monochromatic light that is directed at a sample is elastically scattered. However, in certain circumstances inelastic, Raman, scattering occurs. Raman scattering occurs when the monochromatic light incident on the sample is scattered back at either a higher or lower energy level than the initial energy level of the incident monochromatic light.

An increase in the energy level of scattered monochromatic light occurs when a molecule imparts some of its vibrational energy to the incident monochromatic light that is scattered. Subsequently, a decrease in the energy level of scattered monochromatic light occurs when a molecule absorbs energy from the incident monochromatic light, as vibrational energy. These increases and decreases of the energy level of scattered monochromatic light produce spectra that relate to the vibrations within molecules present in a sample. Analysis of a sample's spectrum, where Raman scattering has occurred, enables the identification of molecules present in a sample, and their concentrations.

Improvements to devices that indicate blood sugar levels of a person are desired. Speed and accuracy in determination of blood sugar levels of diabetics allows for optimal management of their blood sugar levels. Therefore, there is always a need to improve the functionality, accuracy and precision of devices that can be used to determine blood sugar levels.

WO 2006/061565 A1 describes a method and device that uses spatially offset Raman spectroscopy to measure the composition of bone in vivo.

GB 2541110 A describes a device that also uses spatially offset Raman spectroscopy. The disclosed device utilizes a rotatable prism in the optical path.

US-A-2014/171759 discloses an apparatus and method for non-invasive determination of hydration, hydration state, total body water or water concentration by quantitative spectroscopy. The apparatus is able to include non-invasive hydration measurement that can be combined with additional analyte measurement including measurement of glucose. The additional analyte measurement is obtained from the inclusion of additional sensors.

As is mentioned in WO 2018/103943 A1, miniaturisation of the device enables a user to keep the device on their person, which in turn enables them to test their blood sugar levels quickly and easily and whenever necessary. This is particularly important and useful for those with conditions such as diabetes who need regularly to have knowledge of their blood sugar levels.

According to a first aspect of the present invention, there is provided apparatus suitable for non-invasive in vivo measurement by Raman spectroscopy of analyte presence and/or concentration, such as glucose, in the skin of a subject or for Raman spectroscopy of a sample other than human skin such that it has use other than for non-invasive in vivo measurement by Raman spectroscopy of analyte presence and/or concentration, such as glucose, the apparatus comprising; at least one detector; one or more of vertical-cavity surface-emitting lasers spatially distributed around the at least one detector, for irradiating a sample such as the skin of a subject; wherein the at least one detector is configured to receive Raman scattered radiation transmitted from the sample in response to the received radiation from the vertical-cavity surface-emitting lasers.

The applicant has recognised that surprisingly the properties of a VCSEL make them particularly suitable for use in apparatus for non-invasive in vivo measurement, by Raman spectroscopy, of glucose present in interstitial fluid in the skin of a subject. The detector may be a suitable element or component for receiving and/or detecting the Raman scattered radiation emitted in response to incident light from the VCSELs.

In an embodiment, the at least one detector is surrounded by a plurality of VCSELs. In an embodiment, the at least one detector is surrounded by at least one ring of vertical-cavity surface-emitting lasers, i.e. it is arranged within the at least one ring of VCSELs. In another example one or more lines or linear arrays of VCSELs are arranged separated by some distance from the detector.

In an embodiment, the at least one detector is surrounded by a plurality of concentric rings of radiation sources. In this preferred embodiment, a plurality of rings of optical sources, such as VCSELs are provided. In an apparatus for in vivo measurement of analyte concentration this is particularly advantageous as it provides for the easy and repeatable selection of analysis depth, without requiring moving parts in a probe or apparatus itself. In other words, different rings or groups of optical sources e.g. VCSELs, can be arranged such that when activated they irradiate a region some determined distance from the sources or the probe if contained within a probe. For example, if the sources are arranged at an end surface or near a surface that in use will engage with a user's skin, by activating different subgroups of the sources in the apparatus the resultant point of interrogation, i.e. the point to which the optical radiation is directed can be controllably varied.

As used herein “ring” clearly is not limited to (although it does include) a geometrical circle. The rings can be square, elliptical, triangular or any other shape that generally surround the detector.

In an embodiment, the at least one detector is surrounded by a plurality of rings of vertical-cavity surface-emitting lasers.

In an embodiment, the apparatus comprises a plurality of detectors surrounded by a common ring of vertical-cavity surface-emitting lasers.

In an embodiment, the apparatus comprises a plurality of detectors surrounded by shared rings of vertical-cavity surface-emitting lasers.

In an embodiment, the vertical-cavity surface-emitting lasers are configured to provide at least two different wavelengths of radiation to irradiate a sample.

In one example, the detector(s) and source(s) are arranged such that they are placed in use on the same side of a user's skin under investigation. In another example, the detector(s) and source(s) are arranged such that they are placed in use on the opposite side of a user's skin under investigation.

In an embodiment, the apparatus includes a temperature sensor to control or monitor the VCSEL temperature. Knowledge of the VCSEL temperature is enables the recorded spectra to be adjusted in accordance with the excitation wavelength (due to the relationship between VCSEL temperature and VCSEL wavelength).

In an embodiment, the apparatus further includes means for temperature stabilization of the VCSELs such as, say, a thermoelectric cooler, so as to avoid excitation wavelength drift.

In one example, with or without use of temperature stabilization, the excitation wavelength is tracked by use of a spectrometer. If drift is detected the recorded spectra can be adjusted in dependence on the excitation wavelength. The excitation wavelength can be stabilized by control of VCSEL temperature and/or the applied driving current or signal.

According to a second aspect of the present invention, there is provided apparatus for non-invasive in vivo measurement, by Raman spectroscopy, of analyte presence and/or concentration, such as glucose, in a sample such as the skin of a subject, or for Raman spectroscopy of a sample other than human skin such that it has use other than for non-invasive in vivo measurement by Raman spectroscopy of analyte presence and/or concentration, such as glucose, the apparatus comprising; at least one radiation source, for irradiating a sample such as a the skin of a subject; and a plurality of detectors spatially distributed around the radiation source, wherein the plurality of detectors are configured to receive Raman scattered radiation from the sample in response to the received radiation from the at least one radiation source.

In an embodiment, the at least one radiation source is a vertical-cavity surface-emitting laser.

In an embodiment, the at least one radiation source is surrounded by at least one ring of detectors. In another example one or more lines or linear arrays of detectors are arranged separated by some distance from the radiation source.

In an embodiment, the at least one radiation source is surrounded by a plurality of rings of detectors.

In an embodiment, the apparatus comprises a plurality of radiation sources surrounded by at least one ring of detectors.

In an embodiment, the apparatus comprises a plurality of radiation sources surrounded by a shared plurality of rings of detectors.

In an embodiment, the radiation sources are configured to provide at least two different wavelengths of radiation to irradiate a sample.

In an embodiment, the apparatus further comprises an analysis unit configured to analyse the detected Raman spectrum and infer glucose levels within a sample.

In an embodiment, the analysis unit is further configured to eliminate background radiation and highlight the Raman spectrum of a sample.

In an embodiment, the apparatus comprises a focusing device for focusing the spectrum of Raman scattered radiation transmitted back from the sample for detection.

In an embodiment, the focusing device comprises of at least one optical lens.

In an embodiment, the at least one optical lens is a convex lens.

In an embodiment, the focusing device comprises a plurality of optical lenses.

In an embodiment, the plurality of optical lenses comprises a plurality of convex and/or concave lenses.

In an embodiment, a fibre or fibre bundle is used to receive the Raman scattered radiation transmitted back from the sample.

In an embodiment, the focusing device comprises at least one mirror.

According to a third aspect of the present invention, there is provided a method for non-invasive in vivo measurement, by Raman spectroscopy, of analyte presence and/or concentration, such as glucose, in the skin of a subject or for Raman spectroscopy of a sample other than human skin such that it has use other than for non-invasive in vivo measurement by Raman spectroscopy of analyte presence and/or concentration, such as glucose, the method comprising; using the apparatus of any of the previous claims to detect and measure the spectrum of Raman scattered radiation from a sample such as the skin of a subject; and analysing the spectrum of the detected Raman scattered radiation to determine the presence and/or concentration of analyte in the sample such as the skin of a subject.

In an embodiment, the method comprises controlling the vertical-cavity surface-emitting lasers to vary collection depth of the Raman scattered radiation.

In an embodiment, the method comprises executing an algorithm to determine the Raman spectrum in dependence on the respective positions of the at least one radiation source and at least one detector relative to the position of the sample.

In an embodiment, the method comprises executing the algorithm to eliminate background fluorescence.

In an embodiment, the algorithm utilizes Shift-Excitation Raman Difference Spectroscopy (SERDS).

In an embodiment, the method comprises: eliminating non-Raman background fluorescence by comparing the shifts in spectral peaks of observed scattered radiation from a sample, irradiated by at least two different wavelengths; removing spectral features, such as spectral peaks, that do not shift between the spectra created by the at least two difference wavelengths of radiation; and analysing remaining spectral peaks, for the presence of analyte within the sample.

According to a fourth aspect of the present invention, there is provided apparatus for non-invasive in vivo measurement, by Raman spectroscopy, of analyte presence and/or concentration, such as glucose, in the skin of a subject, the apparatus comprising; at least one detector a radiation source for irradiating the skin of a subject, spatially distributed around the at least one detector; wherein the at least one detector is configured to receive a spectrum of Raman scattered radiation transmitted back from the sample in response to the received radiation from the radiation source. The apparatus is also suitable for Raman spectroscopy of a sample other than human skin such that it has use other than for non-invasive in vivo measurement by Raman spectroscopy of analyte presence and/or concentration, such as glucose.

According to a further aspect of the present invention, there is provided apparatus for non-invasive in vivo measurement, by Raman spectroscopy, of glucose present in interstitial fluid in the skin of a subject, the apparatus comprising; a plurality of radiation sources, for irradiating a sample in the skin of a subject; and at least one detector; wherein the plurality of radiation sources are spatially distributed around the at least one detector; and wherein the at least one detector is configured to receive a spectrum of Raman scattered radiation transmitted back from the sample in response to the received radiation from the at least one radiation source. The apparatus is also for Raman spectroscopy of a sample other than human skin such that it has use other than for non-invasive in vivo measurement by Raman spectroscopy of analyte presence and/or concentration, such as glucose.

In an embodiment, the plurality of radiation sources are vertical-cavity surface-emitting lasers. As is known, a VCSEL is a laser that generates beam emission perpendicular to a top surface, contrary to conventional edge-emitting semiconductor lasers which emit from surfaces formed by cleaving an individual chip out of a semiconductor wafer. The applicant has recognised that surprisingly the properties of a VCSEL make them particularly suitable for use in apparatus for non-invasive in vivo measurement, by Raman spectroscopy, of glucose present in interstitial fluid in the skin of a subject.

In an embodiment, the at least one detector is surrounded by at least one concentric ring of radiation sources. The dimensions and vertical emission surface of a VCSEL makes them particularly suitable for use in an arrangement such as a ring of optical sources to provide incident light in an apparatus for non-invasive in vivo measurement, by Raman spectroscopy, of glucose or other present in the skin of a subject.

Again, as used herein “ring” clearly is not limited to (although it does include) a geometrical circle. The rings can be square, elliptical, triangular or any other shape that generally surround the detector. As mentioned above, in another example one or more lines or linear arrays of radiation sources such as VCSELs can be used separated from the detector.

In an embodiment, there are a plurality of detectors that are surrounded by at least one concentric ring of radiation sources.

In an embodiment, there are a plurality of detectors that are surrounded by a plurality of concentric rings of radiation sources.

In an embodiment, the radiation sources are configured to provide at least two different wavelengths of radiation to irradiate a sample.

According to a further aspect of the present invention, there is provided apparatus for non-invasive in vivo measurement, by Raman spectroscopy, of glucose present in interstitial fluid in the skin of a subject (or for other uses as in the aspects mentioned above), the apparatus comprising; at least one radiation source, for irradiating a sample in the skin of a subject; and a plurality of detectors; wherein the plurality of detectors are spatially distributed around the at least one detector; and wherein the plurality of detectors are configured to receive a spectrum of Raman scattered radiation transmitted back from the sample in response to the received radiation from the at least one radiation source.

In an embodiment, the at least one radiation source is a vertical-cavity surface-emitting laser. The applicant has recognised that surprisingly the properties of a VCSEL make them particularly suitable for use in apparatus for non-invasive in vivo measurement, by Raman spectroscopy, of glucose present in interstitial fluid in the skin of a subject.

In an embodiment, the at least one radiation source is surrounded by at least one concentric ring of detectors. Each of the detectors may be simply an optical interface arranged to receive radiation and couple it onwards for analysis, and/or they could be a photosensitive component such as a photodiode or a component of a CCD to determine the intensity and wavelength of incident light. In the case of an optical interface, they can include one or more filters as required or desired.

In an embodiment, the at least one radiation source is surrounded by a plurality of concentric rings of detectors. As above, “ring” clearly is not limited to (although it does include) a geometrical circle. The rings can be square, elliptical, triangular or any other shape that generally surround the optical source.

In an embodiment, there are a plurality of radiation sources that are surrounded by at least one concentric ring of detectors.

In an embodiment, there are a plurality of radiation sources that are surrounded by a plurality of concentric rings of detectors.

In an embodiment, the radiation sources are configured to provide at least two different wavelengths of radiation to irradiate a sample.

In an embodiment, the apparatus further comprises an analysis unit configured to analyse the detected Raman spectrum and infer glucose levels within a sample.

In an embodiment, the analysis unit is further configured to eliminate background radiation and highlight the Raman spectrum of a sample. A filter such as a Rayleigh filter is preferably used for this purpose.

In an embodiment, there is a focusing device for focusing the spectrum of Raman scattered radiation transmitted back from the sample for detection. In an embodiment, the focusing device comprises of at least one optical fibre.

In an embodiment, the focusing device is comprised of at least one optical lens.

In an embodiment, the at least one optical lens is a convex lens.

In an embodiment, the focusing device is comprised of a plurality of optical lenses.

In an embodiment, the plurality of optical lenses is a plurality of convex and/or concave lenses.

In an embodiment, the focusing device is comprised of at least one mirror.

In an embodiment, the at least one mirror is a concave mirror.

In an embodiment, the focusing device is comprised of a plurality of mirrors.

According to a further aspect of the present invention, there is provided a method for non-invasive in vivo measurement, by Raman spectroscopy, of glucose present in interstitial fluid in the skin of a subject (or for other uses as in the aspects mentioned above), the method comprising; using the apparatus of any of the previous claims to detect and measure the spectrum of Raman scattered radiation from a sample in the skin of a subject; and analysing the spectrum of the detected Raman scattered radiation to determine the concentration of glucose present in the interstitial fluid in the skin of a subject.

In an embodiment, an algorithm is used to improve the accuracy and precision of the analysis of the spectrum of the detected Raman scattered radiation based on the respective positions of the at least one radiation source and at least one detector relative to the position of the sample.

In an embodiment, the algorithm also applies a technique for fluorescence background elimination.

In an embodiment, the technique for fluorescence background elimination eliminates non-Raman background fluorescence by comparing the shifts in spectral peaks of observed scattered radiation from a sample, irradiated by at least two different wavelengths of radiation, and removing any spectral peaks that do not shift between the spectra created by the at least two difference wavelengths of radiation, and to analyse the remaining spectral peaks, that shifted, for the presence of glucose within a sample.

In WO 2006/061565 A1 there is no discussion of spatially distributing radiation sources around the detector, or spatially distributing detectors around the radiation source.

GB 2541110 A does not discuss spatially distributing radiation sources around the detector, or spatially distributing detectors around the radiation source.

Accordingly, the present system and method provides the benefits of spatially offset Raman spectroscopy, but in a compact device that is more convenient to a user as it allows them to check their blood sugar levels when necessary, throughout the day, wherever they may be.

According to a further aspect of the present invention, there is provided apparatus for non-invasive in vivo measurement, by Raman spectroscopy, of analyte presence and/or concentration, such as glucose, in the skin of a subject (or for other uses as in the aspects mentioned above), the apparatus comprising; at least one detector; a controllable VCSEL radiation source spaced from the at least one detector, for irradiating the skin of a subject with light, and being configured to selectively change the wavelength of the light in accordance with a SWEPT Source Raman methodology; a bandpass filter to receive Raman scattered radiation transmitted back from the sample; a processor to generate a Raman spectrum from the received Raman scattered radiation.

In the current applicant's International application number WO2011/83111 (granted in many jurisdictions) there is described a method and apparatus for non-invasive in vivo measurement by Raman spectroscopy of glucose present in interstitial fluid in skin. Amongst other aspects there is described apparatus for non-invasive in vivo measurement by Raman spectroscopy of glucose present in interstitial fluid in the skin of a subject, comprising a light source, optical components defining a light path from said light source to a measurement location, a light detection unit, optical components defining a return path for Raman scattered light from said measurement location to said light detection unit, and a skin engaging member having a distal surface for defining the position of said optical components defining the return path with respect to a surface of said skin in use, and wherein said optical components defining a return path for Raman scattered light selectively transmit to said light detection unit light scattered from near said measurement location such that at least 50% of Raman scattered light received at the light detection unit originates at depths from 60 to 400 μm beyond said distal surface of the skin engaging member.

There is a desire to further miniaturise the spectrometer probe used in such devices.

According to a further aspect of the present invention, there is provided apparatus for in vivo measurement, such as non-invasive in vivo measurement, by Raman spectroscopy of analyte presence and/or concentration, such as glucose, in the skin of a subject, the apparatus comprising; a spectrometer having a slit for receiving a Raman spectrum from a sample; an integrated probe for coupling to the spectrometer, wherein the probe is of generally planar configuration.

In an example, the integrated probe comprises a PCB having arranged thereon plural optical sources and arranged around the slit of the spectrometer.

In an example, the plural optical sources are VCSELs.

In an example, the PCB comprises a window and the optical sources are arranged around the window.

In an example, plural rows of VCSELs are provided on either side of the window.

In an example, the apparatus comprises directing optics to control the distance of the focal point of the optical sources from the plane of the planar integrated probe.

In an example, the apparatus comprises source optics arranged for controlling the transmission of light from the optical sources.

According to a further aspect of the present invention, there is provided an integrated probe for coupling to a spectrometer, for in vivo measurement, such as non-invasive in vivo measurement, by Raman spectroscopy of analyte presence and/or concentration, such as glucose, in the skin of a subject, wherein the probe is of generally planar configuration.

There is also a desire to have a Raman probe that is compact and easy to use in varied technical fields.

According to a further aspect of the present invention, there is provided apparatus for Raman spectroscopy, the apparatus comprising; a spectrometer having a slit for receiving a Raman spectrum from a sample; an integrated probe for coupling to the spectrometer, wherein the probe is of generally planar configuration.

According to a further aspect of the present invention, there is provided a Raman probe for provision of Raman derived radiation to a spectrometer, the probe being an integrated probe for coupling to a spectrometer, wherein the probe is of generally planar configuration. Preferably the probe comprises a PCB having one or more VCSELs or other light sources formed thereon and controlled to radiation to a sample. The PCB preferably has a slit, which in use may be arranged in alignment with a spectrometer entrance slit. The VCSELs or optical sources are preferably arranged adjacent to the longitudinal sides of the slit. The VCSELs or other optical sources are preferably controlled or controllable to operate in accordance with the SORS methodologies described herein.

Embodiments of the present invention will now be described in detail with reference to the accompanying drawings, in which:

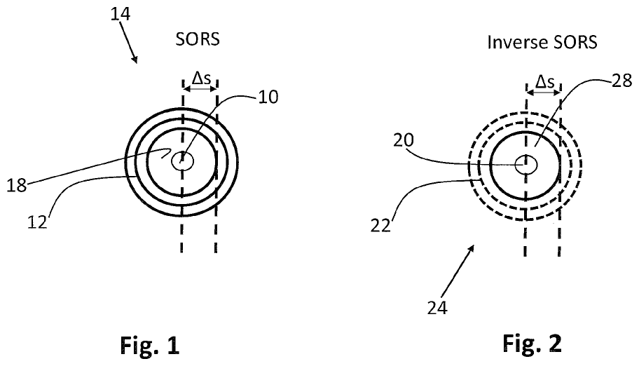

FIG. 1 shows a schematic view of a first example of an optical arrangement for use in a device for non-invasive in vivo measurement of analyte present in the skin of a subject;

FIG. 2 shows a schematic view of a second example of an optical arrangement for use in a device for non-invasive in vivo measurement of analyte present in the skin of a subject;

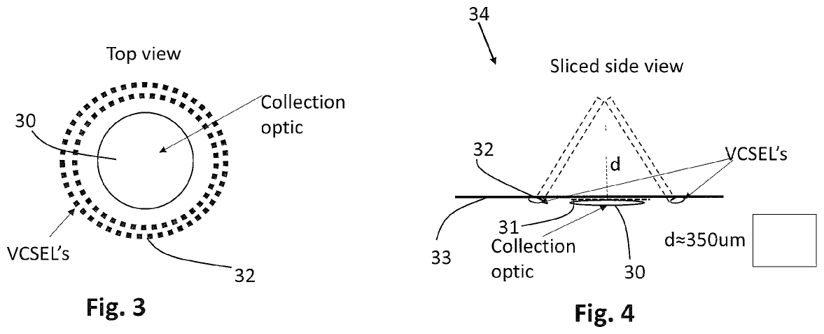

FIG. 3 is a schematic plan view of a further example of an optical arrangement for use in a device for non-invasive in vivo measurement of analyte present in the skin of a subject;

FIG. 4 is a side view of the arrangement of FIG. 3;

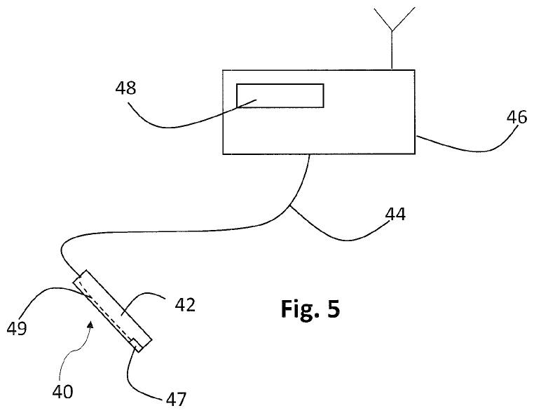

FIG. 5 is a schematic view of a probe assembly incorporating the arrangement of any of FIGS. 1 to 4:

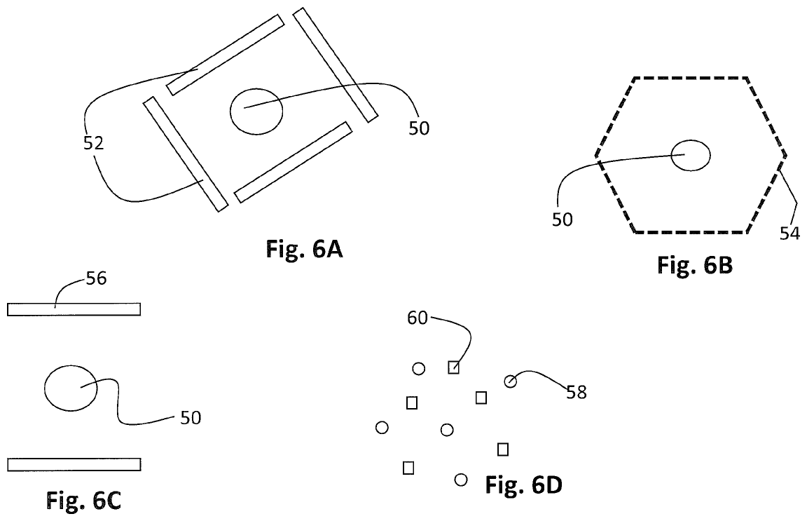

FIGS. 6A to 6D show schematically alternative configurations for optical detectors and sources of an optical arrangement for use in a device for non-invasive in vivo measurement of analyte present in the skin of a subject;

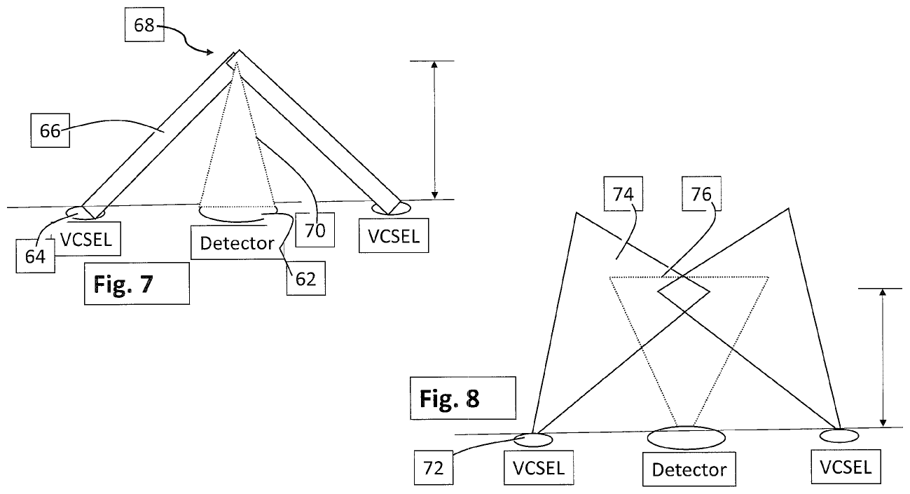

FIGS. 7 to 13 show schematically alternative configurations for optical detectors and sources of an optical arrangement for use in a device for non-invasive in vivo measurement of analyte present in the skin of a subject.

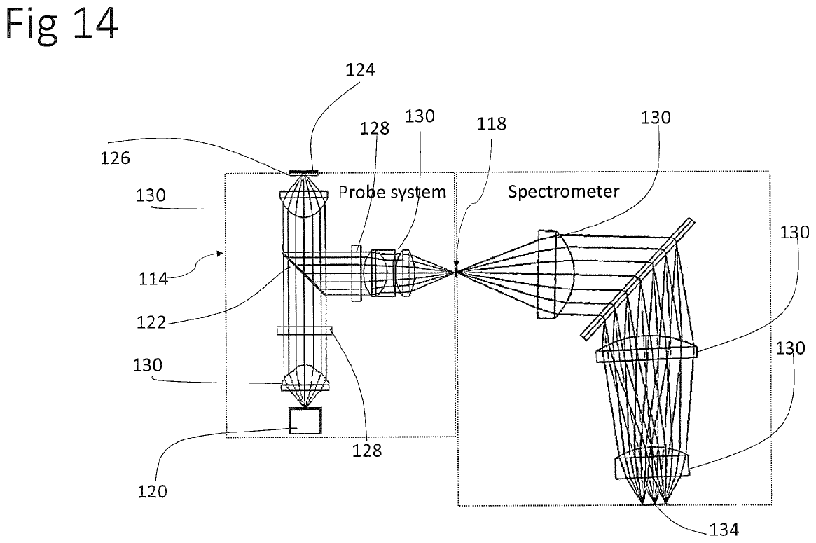

FIG. 14 is a schematic representation of a known optical probe, connected to a known spectrometer;

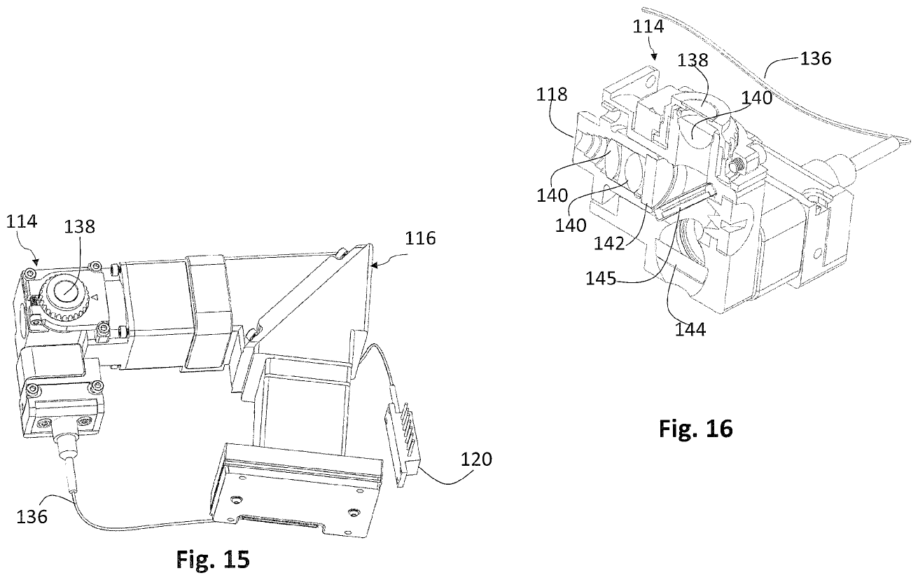

FIG. 15 is a schematic representation of a known optical probe system;

FIG. 16 is a cross section through a part of the probe system of FIG. 15;

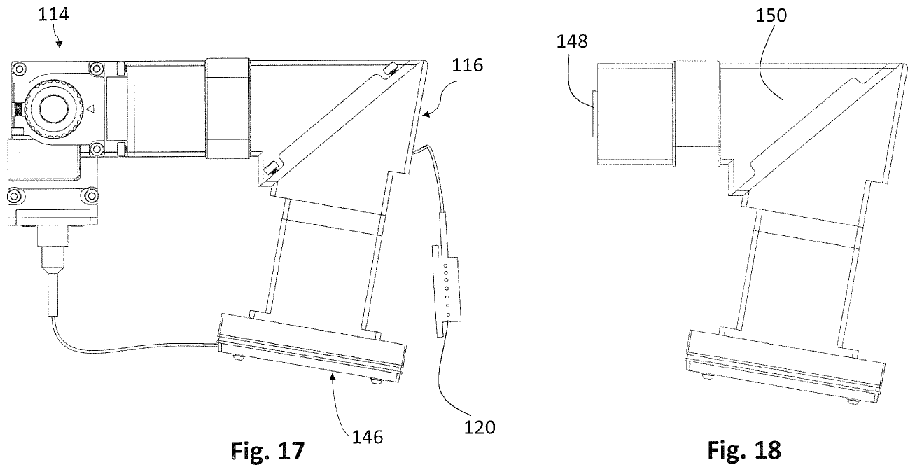

FIG. 17 is a plan view of the probe system of FIG. 15;

FIG. 18 is a plan view of a known spectrometer combined with a probe according to a current embodiment;

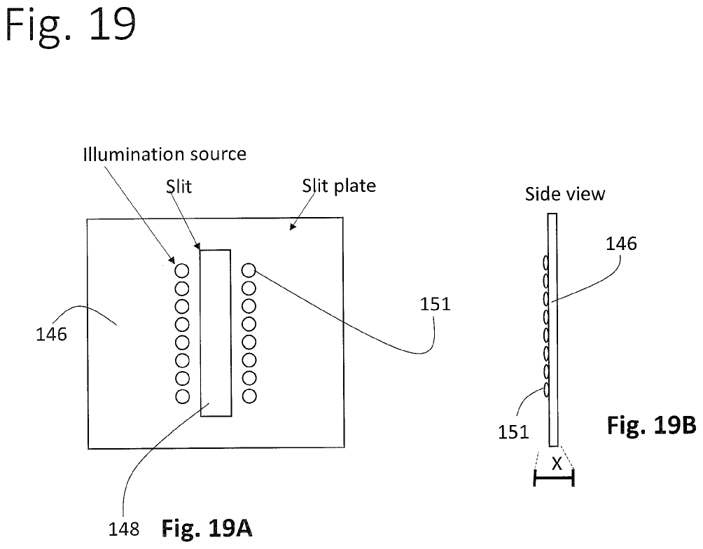

FIGS. 19A and 19B show a plan view and a side view respectively of an optical probe interface;

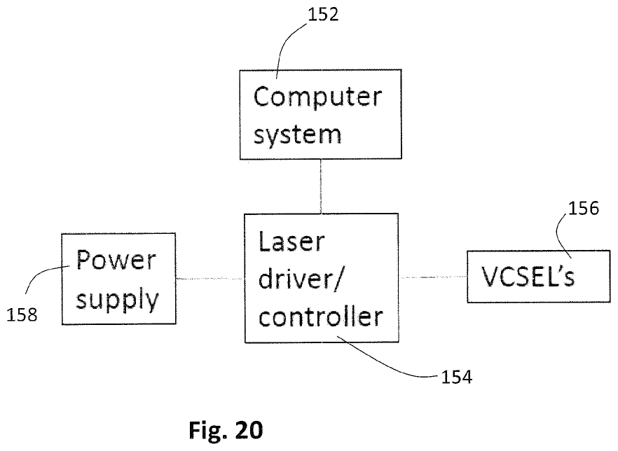

FIG. 20 shows a schematic block diagram of an optical control system for use in an optical probe;

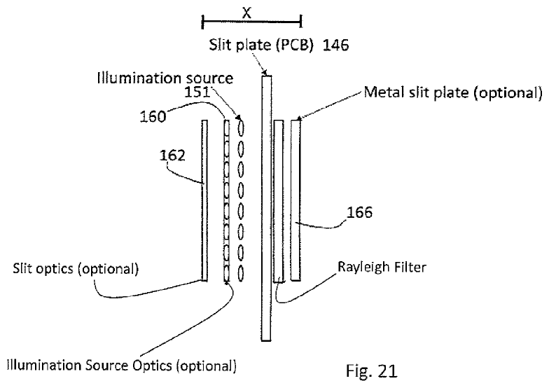

FIG. 21 shows an exploded cross sectional view through the probe system.

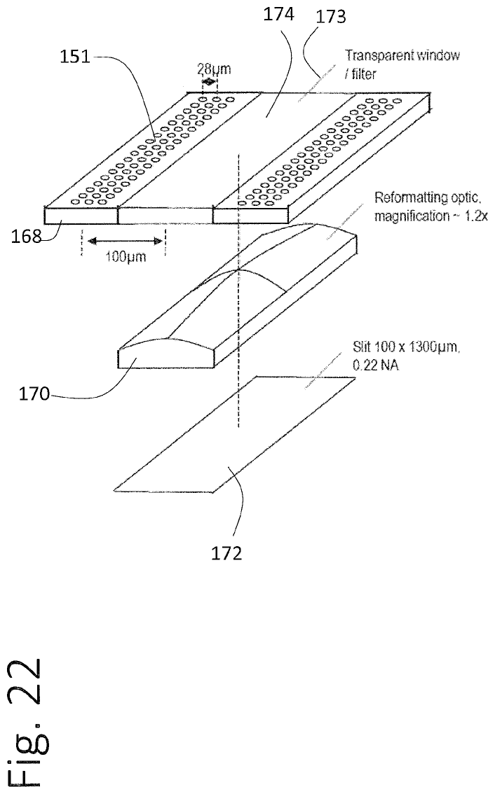

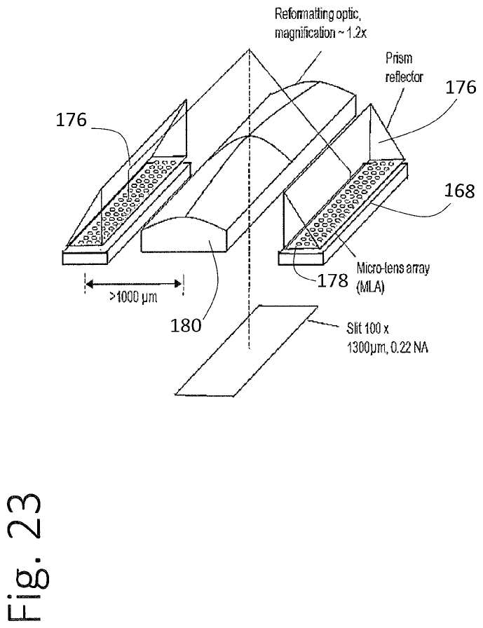

FIGS. 22 and 23 show schematic views of optical interfaces according to embodiments;

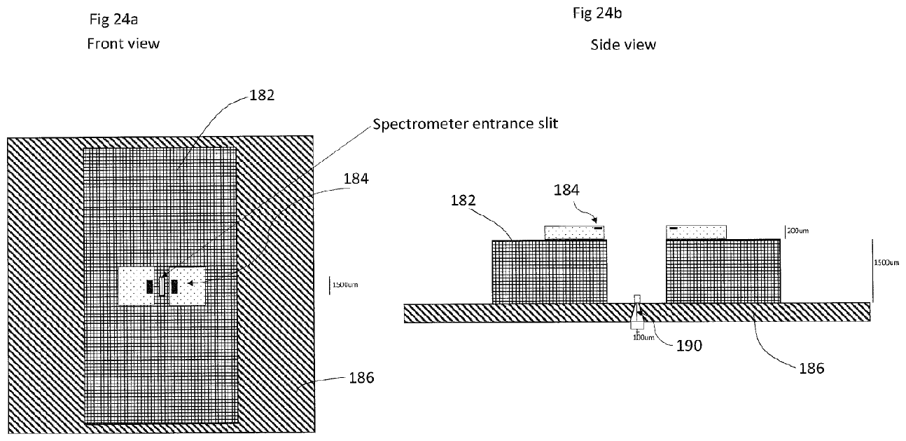

FIGS. 24A and 24B show respectively a plan view and a sectional view of an exemplary probe; and

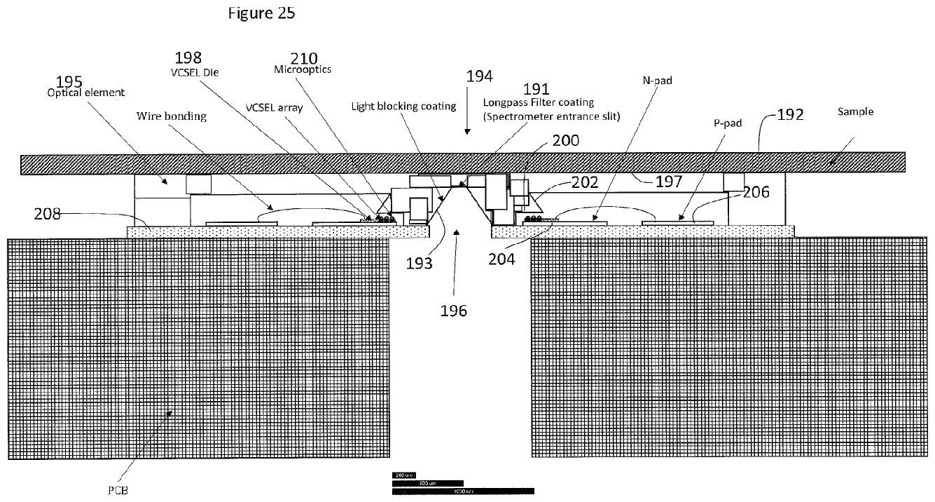

FIG. 25 shows a cross sectional view of an exemplary probe.

### DETAILED DESCRIPTION

FIG. 1 shows a schematic view of a first example of an optical probe arrangement for use in a device for non-invasive in vivo measurement of analyte present in the skin of a subject. Typically, the arrangement can be used for measuring the concentration of glucose in the interstitial fluid in a user's skin.

The arrangement shown will typically be provided as part of a system, described in general below with reference to schematic view of FIG. 5, that includes a spectrometer, a processor and some means of generating an output for a user. Referring to FIG. 1, an optical source 10 is provided surrounded by multiple rings of Raman detectors 12. The arrangement may generally be considered a Spatially Offset Raman Spectroscopy (SORS) 14 since the Raman detectors 12 and optical source 10 are spatially offset.

The arrangement 14 will typically be provided as part of a probe, as shown in FIG. 5 below. FIG. 1 is looking end-on to the surface of the arrangement or probe that will in use be brought into contact with the skin of a user. The probe will typically have a region 18 that provides an interface for optical source 10 to illuminate the subject and defines an offset between the optical source 10 and the detectors 12. A Raman signal is transmitted from the subject and is captured by the Raman detectors 12. The detected Raman scattered signal can then be analysed to produce an output Raman spectrum, from which an indication of the presence and/or concentration various analytes, such as glucose, can be obtained.

The laser source 10 of the SORS arrangement 14 is most preferably a vertical-cavity surface-emitting laser (VCSEL) which enables miniaturisation of the device without any loss of functionality.

The detectors 12 of the SORS arrangement 14 are a means for communicating the detected Raman signal to one or more spectrometers or in combination work as spectrometers. A spectrometer is an optical apparatus that works by separating the light beam directed into the optical apparatus into different frequency components and subsequently measuring the intensity of these components by using analysis devices such as CCD detectors, CCD arrays or any other suitable light capturing device.

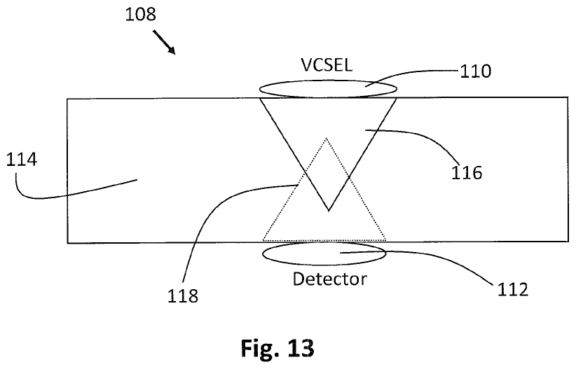

The detectors 12 are shown in the example of FIG. 1 as a series of concentric circular rings 12. Other arrangement can be used and the detectors need not be circular or even ring shaped. In one embodiment the detectors can be provided as elliptical or square on shape, or indeed any configuration that provides the detectors as separated from the optical source 10. Typically, the detector(s) and source(s) are arranged such that they are placed in use on the same side of a user's skin under investigation. Other configurations are also possible as explained below with reference to FIG. 13.

In another example, one or more detector strips may be provided, e.g. two parallel detector strips on either side of the optical source 10 are provided. The, or each of the, detector strips may be provided as straight line linear detector strips or curved detector strips, each provided some separation from the optical source 10. One could be curved and another straight. It will be appreciated that the detectors 12 can function simply as receivers arranged to receive and couple the Raman signal onwards for analysis. The detectors 12 could include circuits or componentry to enable the detectors themselves to determine the spectrum from the received radiation.

FIG. 2 shows an alternative embodiment, wherein there is a Raman detector 20 surrounded by multiple optical sources laid out in this example generally as rings 22 of optical sources. The optical sources may be lasers in the form of VCSELs. Again, although the rings 22 of optical sources are shown as a series of concentric circular rings 12, other arrangements can be used and the optical sources need not be arranged in circular or even ring-shaped configuration. In one embodiment the optical sources detectors can be provided as elliptical or square on shape, or indeed any configuration that provides the sources are separated from the detector 20. As used with reference to the plurality of sources it will be appreciated that the term rings refers to the general layout of the plurality of sources. The plurality of sources could be laid out in other configurations too such as a two dimensional array and/or in the form of parallel lines of optical sources.

The arrangement 24 will typically be provided as part of a probe, as shown in FIG. 5 below. The view of the arrangement of FIG. 2 is looking end on to the surface of the arrangement or probe that will in use be brought into contact with the skin of a user. Typically, the arrangement will be provided within or as part of a probe having a casing (not shown in FIG. 2, but visible in the schematic view of FIG. 5) with an offset or region 28 that provides access for the detector 20 to receive transmitted Raman radiation from a user's skin. The illuminated sample of the subject then is a source of Raman scattered light which is received by the detector 20.

The detector 20 could be the end face of an optical fibre or an optical fibre bundle comprising multiple fibres. Preferably some optical arrangement such as a lensing arrangement is provided on the end of the fibre or fibre bundle to communicate the received light into the fibre bundle for onward coupling to a processor or spectrometer, as described above with reference to FIG. 1.

The Raman detector 20 of the inverse SORS arrangement 24 is preferably a spectrometer or is coupled to one to enable a determination of the Raman spectrum to be made.

The rings of laser sources 22 of the inverse SORS device 24 can include any suitable form of laser emitting device. However, to improve the miniaturisation of the device the laser sources 22 are preferably VCSELs. Typically, the dimension of the probe and or arrangement is such that it is easily and ergonomically usable by an individual. In practice, the diameter of the outer rings shown in each of FIGS. 1 and 2 will be between 0.1 and 2 cm.

FIGS. 3 and 4 show a further embodiment combining the inverse SORS device 24 of FIG. 2 with a focusing device functioning as a detector unit 30. The focusing device or detector unit 30 is surrounded by rings of VCSELs 32. The detector unit 30 provides a means for focusing the received Raman scattered light, possibly onto an upstream component, for analysis of blood glucose levels in the sample of a subject.

FIG. 4 shows a side view of the embodiment of FIG. 3. In FIG. 4 the arrangement including the lens 30 is arranged in contact with a user's skin 33. VCSELs 32 are arranged to irradiate a region at a position, in this non-limiting example, approximately 350 micrometres under the surface of (or rather, within) the user's skin. A filter 31 is provided generally at the input to the detector 30. Such a filter may optionally be provided in any or all of the described examples, but is only shown in FIG. 3.

The optical arrangement described herein provides a number of advantages. The use of VCSELs facilitates the collection of Raman signal from a larger volume which means that the system as a whole is less sensitive to skin variation, such as skin thickness variation. In the example in which a plurality of VCSELs are provided, preferably in rings of some shape, it is possible to vary collection depths without actually having to move anything within the probe. Simply activating a different selection of the VCSELs will stimulate Raman signal within a user at different locations or depths. Finally, the use of VCSELs enables the reduction in the probe of other optical elements such as focusing hardware and the like.

The lens 30 is arranged to receive Raman scattered radiation generated by the incident radiation from the VCSELs 32 and focus it for onward transmission to a detector or a spectrometer for further analysis.

The focusing device 30 is preferably but not limited to being a collection optic which refracts the received Raman scattered light for onward transmission. The focusing device 30 may also include or consist of one or more of a mirror, a group of interconnected mirrors, an array of collection optics, or a combination of mirrors and collection optics, and filters.

In order to infer the concentration of glucose in the sample of a subject the openings of any of the previous arrangements 14, 24, or 34 in combination with the features of FIGS. 3 and 4, is applied the surface of skin the subject chooses as a sample. The device then emits laser light onto the sample which Raman scatters the laser light back to the Raman detector 12, or Raman detector unit 20.

The Raman detectors 12 or Raman detector unit 20 then communicates the received Raman signal onwards for analysis of the spectra received for the presence of glucose in the sample, and provides an indication of, say, the blood glucose level in the sample to the subject.

Referring to FIG. 5 an overall assembly is shown incorporating an arrangement like any of those shown in FIGS. 1 to 4. A probe 40 is provided having an outer casing defining a handle 42 shaped for an operator to hold when using the system. Cabling 44 is provided coupling the probe 40 to a processing unit 46 such as a general purpose computer running particular software or a dedicated hardware unit. The cabling 44 may be optical, electrical or both and serves to communicate signals or data between the probe 40 and the processing unit 46. In place of (or as well as) cabling, a wireless connection may be used between the probe 40 and the processing unit 46.

Preferably the processing unit 46 includes a display 48 which functions as a GUI to indicate a reading or result to a user when a test is done using the system. In one example the processing unit is entirely electrical without optical functionality. The optical componentry and processing is all integrated and incorporated within the probe 40. This is achievable due to the use of VCSELs enabling miniaturisation of the optics. Thus, the cabling 44 is electrical, communicating control signals and data between the probe 40 and the processing unit 46.

In another example, the probe 40 includes VCSELs but the spectrometer or CCD devices that might be used are housed within the processing unit, such that the cabling 44 includes one or more optical fibres as well as electrical cabling for power and/or signalling.

In the example shown, a temperature sensor (or sensors) 47 is provided as part of the probe 40. The temperature sensor 47 is coupled to the processing unit 46 via conductor 49. The temperature sensor is arranged and configured to measure the temperature of the VCSELs provided within the probe, and preferably arranged to couple the measured temperature to the controller 46. If required, the VCSEL temperature is mapped/converted to an excitation wavelength and if necessary, the recorded spectra are adjusted in accordance with the excitation wavelength.

Furthermore, in an example, temperature stabilization of the VCSELs is enabled by use of, say, a thermoelectric cooler, so as to avoid excitation wavelength drift.

In one example, with or without use of temperature stabilization, the excitation wavelength is tracked by use of a spectrometer. If drift is detected the recorded spectra can be adjusted in dependence on the excitation wavelength. The excitation wavelength can be stabilized by control of VCSEL temperature and/or the applied driving current or signal.

In an embodiment an algorithm is used to analyse the received Raman spectrum to determine the concentration of glucose or some other analyte. If the signal comes from the skin it is likely that it will indicate the concentration of glucose within the interstitial fluid rather than directly in the blood, but this corresponds closely to the level of glucose in the blood albeit with a small time shift. The algorithm, known as dual wavelength shift-excitation Raman Difference Spectroscopy is used. The difference between the two wavelengths is typically less than 5 nm and preferably about 1 nm. The method enables use of a VCSEL probe as described herein arranged to provide background fluorescence elimination. In a general sense this is done with the use of two incident wavelengths. VCSELs are provided having two different transmission wavelengths and due to their small size it is possible to arrange them all within the system as described above with reference to any of FIGS. 1 to 5.

As follows from Kasha's rule, the shift-excitation wavelength for fluorescence background elimination is unaltered for small changes in excitation photon energy, while the generated Raman spectrum does shift according to the excitation photon energy change. Thus, by subtraction of two spectra from each other, acquired with slightly different excitation wavelengths, provides for the elimination of the background florescence while a Raman difference spectrum remains.

In other words, the algorithm for fluorescence background elimination, eliminates non-Raman background fluorescence by comparing the shifts in spectral peaks of observed scattered radiation from a sample, irradiated by at least two different wavelengths of radiation by the laser sources. This enables isolation of the shifted signal, for analysis of the presence of glucose or some other analyte with the sampled volume. In an example this is achieved by providing the optical sources, such as VCSELs, in a distributed way around the detector. Different groups of the individual optical sources are activated such that the target is sequentially irradiated by radiation of the two different wavelengths. In an example where the optical sources are arranged in one or more rings, any one of the one or more rings may be made up of optical sources in which every other optical source has the same transmission wavelength. If three different wavelengths are used, every third optical source will have the same transmission wavelength.

If the optical sources are not arranged in rings, but, say, in a two dimensional array of rows, every other row, may be arranged to have the same transmission wavelength, with intervening rows having some other transmission wavelength. Alternatively, in one example, an even greater degree of variation is achieved in that every other optical source in both X and Y directions is arranged to have the same wavelength and every other optical source to have some common but different, wavelength.

Where, say, plural rings of sources are used, the different rings may be arranged each to have their own different transmission wavelength. Alternatively, in another example, every other ring is arranged to have the same first transmission wavelength, with the intervening rings having some same but different transmission wavelength from the first transmission wavelength.

In a further example, a SWEPT Raman probe is provided using VCSELs as the optical source. An array of VCSELs having a wavelength range of some desired value is provided. The exact number of wavelengths can be varied as per application, but typically a spectral range of, say, 750 to 960 nm, 750 to 860 nm or 850 to 960 nm is provided. A wavelength step is selected and a bandpass filter provided at some value from the original excitation wavelength. The Raman spectrum can then be reconstructed using known SWEPT Raman methodologies

In general, the use of VCSELs facilitates the creation of a SWEPT Raman probe for use in determining in vivo concentrations of analyte in a user's skin.

FIGS. 6A to 6D show schematically alternative configurations for optical detectors and sources of an optical arrangement for use in a device for non-invasive in vivo measurement of analyte present in the skin of a subject.

FIG. 6A shows a configuration in which an optical detector 50 is arranged within a number of linear arrays 52 of VCSEL optical sources. FIG. 6B shows a configuration in which optical detector 50 is arranged within a generally hexagonal continuous array 54 of VCSELs.

FIG. 6C shows an example in which optical detector 50 is arranged between two parallel linear arrays of VCSEL sources 56 and FIG. 6D shows an example in which a number of detectors 58 are distributed in a plane amongst a similarly randomly distributed array of VCSEL sources 60. In each of the examples shown in FIGS. 6A to 6D, it will be appreciated that a detector is provided at some separation from the optical sources in the form of VCSELs. Similar to the general configuration of, say, FIG. 3, the detector or collection optic 50 is arranged within and/or surrounded by the optical sources. Similarly, the configurations shown could be used in an “inverse” manner in which the optical sources are arranged generally in the position of the detectors in FIGS. 6A to 6C and the detector(s) instead arranged to surround the optical sources. Thus, in this configuration an inverse SORS optical arrangement would be provided.

Looking now at FIG. 7, an example of an optical arrangement is shown. The general configuration is similar to the arrangement of, say, FIG. 4 described above. In this example, a detector 62 is provided with VCSEL sources 64 arranged around it. The VCSEL sources are arranged to provide generally parallel beams 66 of light directed at a point 68 which is selected to be at the common focus of the detector 62. Thus, detector 62 typically includes a lens having an acceptance cone 70, i.e. a cone that defines a region such that any light generated within the region and directed towards the detector will have an angle of incidence such that it can be received and detected by the detector. Any Raman signal generated within the detector acceptance cone, and that is directed towards the detector can be received by the detector.

FIG. 8 shows an example in which VCSEL sources 72 are arranged to provide divergent VCSEL beams 74. Again, any Raman signal generated within the detector acceptance cone 76, and that is directed towards the detector, can be received by the detector. It will be appreciated, by comparing FIGS. 7 and 8 that great flexibility is enabled by the present system. Indeed, by providing multiple VCSEL sources arranged around a detector, control of the individual VCSELs provides great flexibility in determination of the region of illumination and thus investigation.

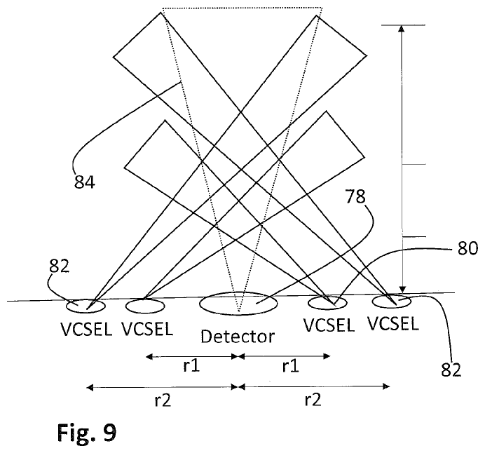

FIG. 9 shows a further example of an optical arrangement for use in a device for non-invasive in vivo measurement of analyte present in the skin of a subject. In this example, a detector 78 is provided. A first and second plurality of VCSELs 80 and 82 are provided. The first plurality of VCSELs 80 is arranged in a ring having a first diameter r1 and the second plurality of VCSELs 82 is arranged in a second ring having a second diameter r2.

A detection cone 84 is shown schematically. Again, as above, Raman signals generated within the detector acceptance cone and that is directed towards the detector can be detected and used to produce the Raman spectrum for the sample.

Each of the VCSELs in the first and second pluralities 80 and 82 are preferably arranged and controlled to provide collimated beams or part-collimated beams and are arranged to be controlled independently. By turning on and off different VCSELs within the first and second pluralities, the Raman signal generated in different volumes within the skin or subject can be collected.

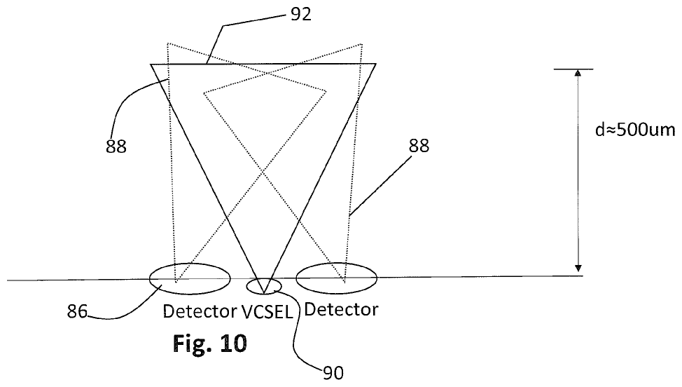

FIG. 10 shows a further configuration for optical detectors and sources of an optical arrangement for use in a device for non-invasive in vivo measurement of analyte present in the skin of a subject. In this example, detectors 86 are provided having detection cones 88. A VCSEL source 90 is provided which typically will comprise a plurality of individual VCSELs. The VCSEL source produces a divergent VCSEL beam 92 thus illuminating a large volume within the skin of the subject. Again, Raman signals generated anywhere within the acceptance cones of the detectors 86, and that is directed towards the detectors, can be detected and used in generation of a Raman spectrum.

FIG. 11 shows a further example of an optical configuration for use in a device for non-invasive in vivo measurement of analyte present in the skin of a subject.

In this example, a number of detectors D1 are provided each having an acceptance cone. The acceptance cones 94 are arranged to intersect the illumination region of a divergent VCSEL source 96. Thus, the use of plural detectors ensures that the signal collected from different areas within the illumination cone 98 of the VCSEL source 96 can be distinguished. Furthermore, understanding can be gained regarding the depth or general location of the optical source due to the use of multiple detectors 97.

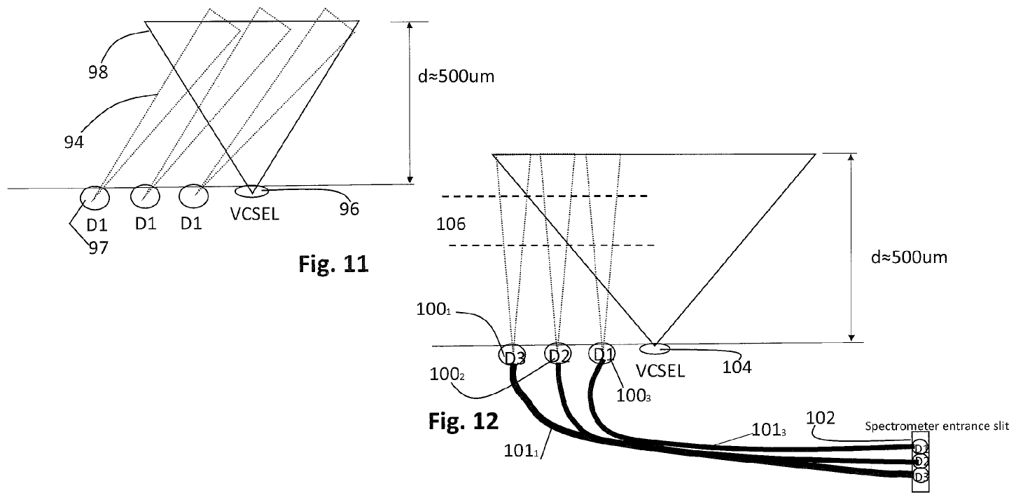

FIG. 12 shows a further example of an arrangement of optical detectors and sources for use in a device for non-invasive in vivo measurement of analyte present in the skin of a subject.

In this example, plural detectors 100₁, 100₂ and 100₃ are provided. An optical connection is provided between each of the detectors and a spectrometer entrance slit 102. The arrangement of the inputs from each of the optical fibres 101 within the spectrometer entrance slit is controlled and fixed such that the spectrum produced by each of the signals from the respective fibres 101₁ to 101₃ can be easily identified.

With the use of a divergent VCSEL source 104 the arrangement can be used to obtain accurate depth information relating to the location origin of a particular spectrum. For example, if the spectrum of D3 is subtracted from the spectrum derived from detector D2 then information regarding the sample within the depth region 106 can be determined. Similarly, other determinations can be made by subtraction of particular pairs of combinations of spectra.

FIG. 13 shows a further example of an arrangement 108 of optical detectors and sources for use in a device for non-invasive in vivo measurement of analyte present in the skin of a subject. In this example, a VCSEL source 110 is provided at a separation from a detector 112. The separation is defined by a sample 114 under investigation being placed between the VCSEL source 110 and the detector 112. The sample could for example be the skin of a subject between the fingers or a pinch of skin taken at some other place on a user's body. The area of illumination 116 of the VCSEL source overlaps with the detector cone 118 of the detector 112.

As explained above, the use of VCSELs or other such similar optical sources enables the miniaturisation of the probe and the use of such methodologies as SORS or inverse SORS. One further particular advantage of the use of optical sources such as VCSELs in an optical probe for the in vivo measurement of analyte concentration is the integration of the VCSELs into or around a spectrometer entrance slit as will now be described in detail.

FIG. 14 is a schematic representation of a known probe for use in a non-invasive system for measuring blood analyte concentration using Raman spectroscopy. Such a probe may for example be used in the in vivo measurement of blood glucose concentrations or the concentrations of other analytes such as alcohol. Such a probe may be of the type generally described in our earlier International applications WO 2018/10394, WO2016034448, and WO2011083111, already referred to above.

The present embodiment provides an integrated probe in which a spectrometer entrance slit is provided and the probe as shown in FIG. 14 and identified as reference numeral 114 therein, that typically comprises plural optical and control components, is provided integrated as part of the assembly. The illumination sources, such as VCSELs are, in effect, provided on the slit. The expression “on the slit” means that the VCSELs are provided in close proximity to the slit of the spectrometer itself. In one example the slit can be provided in a PCB where the VCSELs and optics are provided on the same PCB. Thus, the probe is provided for use in a system, such as a non-invasive system, for measuring blood analyte concentration using Raman spectroscopy. Such a probe may for example be used in the in vivo measurement of blood glucose concentrations or the concentrations of other analytes such as alcohol. The probe is preferably provided as for use in a non-invasive system although it will be appreciated that it can be used in invasive probes or systems as well. It can, for example be used in industrial applications where a Raman spectrometer probe is required. Typical applications include, for example the fields of biochemistry, medicine, agriculture, pharmaceuticals, process control/Quality control, forensic applications and technologies, chemical production, material analysis and environmental monitoring.

Preferably, the illumination sources are provided in the form of VCSELs although other possible illumination sources could also be provided. The illumination can consist of a single source or multiple sources. In a preferred example, the illumination sources comprise paired sources in order to generate an excitation source with specific optical specifications.

As will be described below, optical elements like lenses, optical flats and the like can be placed in front of the illumination sources and/or the spectrometer entrance slit. Such an arrangement including the appropriately sized and configured miniaturised optical components in combination with the optical sources such as the VCSELs still provides for what may be described as an integrated probe. The contrast can be noted markedly in, say, a comparison of FIGS. 17 and 18. In FIG. 17 a conventional probe arrangement is provided in which the probe system 114 is provided coupled to a spectrometer system 116. In FIG. 18, the probe system 148 is effectively provided on a PCB assembly which is arranged generally in contact or fixed to the side of the L-shaped body 150 of the spectrometer.

The illumination sources can consist of two or more individual groups, or single sources, which can be individually controlled. Each group can have specific individual specifications in order to support Raman spectroscopic techniques such as stimulated Raman scattering (SRS), coherent anti-stokes Raman scattering (CARS), shift excitation Raman difference spectroscopy (SERDS) and swept source Raman (SSR) spectroscopy. As discussed herein, the expression “integrated probe” will be used since the probe shown in, say FIG. 14 as a separate physical component including a laser 120, focusing optics (130 etc) and the like is integrated in the probe with the spectrometer.

The illumination sources are preferably arranged in configurations by taking advantage of the spatial offset between the illumination and collection optic based on the SORS principle described above, thereby allowing depth-sensitive probing. Indeed, the operation and control of the integrated probe including the VCSELs or other optical sources can be as shown in and described above with reference to any of FIGS. 1 to 13.

Looking at FIG. 14, a probe system 114 is provided coupled to a spectrometer system 116. The spectrometer entrance slit 118 is the first element in the spectrometer. The probe system 114 is thus the part of the overall system (shown in its entirety in FIG. 14) that provides the controlled light radiation to a sample and receives from the sample a produced Raman signal for onward transmission to the spectrometer 116 in which spectral breakdown and analysis can be performed.

A Raman signal is generated in response to activation of a laser 120 which is directed via optics 122 such as a beam splitter to impinge upon a sample 124. A contact surface 126 may be provided in the form of a transmissive window through which the laser beam travels. The laser beam impinges on the sample 124 and interacts with it generating a Raman spectrum which is transmitted via other optics including a filter 128 and one or more lenses 130 to the spectrometer entrance slit 118. Within the spectrometer, optics 132 are provided which may typically include one or more lenses and/or mirrors and a grating so as to direct the received spectrum onto a detector 134 such as a CCD detector.

The system now to be described integrates the functionality of the probe system 114 into the spectrometer and on or around its slit thereby facilitating miniaturisation and simplification of the apparatus.

Referring to FIGS. 15 and 16, views of a system similar to that shown in FIG. 14 are provided. In FIG. 15, the probe system 114 can be seen coupled to the spectrometer 116. A laser source 120 is provided which through a conduit 136, such as an optical fibre, couples the laser into the probe system 114. An opening 138 is provided which will typically be placed in contact with a user's skin or another sample region for testing.

Referring to FIG. 16, a cross section through the probe system 114 can be seen as can be noted, the system 114 includes various optics in the form of lenses 140 and filter 142. A directing mirror 144 is provided to receive the laser from the conduit 136 and direct it to the sample window 138. A dichroic mirror is provided to direct the generated Raman signal towards the spectrometer slit 118. The precise configuration of the probe system shown either in FIG. 14, or 15 and 16 is merely representative to demonstrate the general scale of the apparatus and the involved complexity.

FIG. 17 shows a plan view of the system of FIG. 15. An imaging device or detector 146 is arranged to receive the Raman spectrum once it has passed through the redirecting optics as described above with reference to FIG. 14.

FIG. 18 shows a plan view of an embodiment of a probe system now to be described. The probe system 114 is replaced by an integrated probe 148 to be described in greater detail below. The generally L-shaped body 150 is the spectrometer as seen in, say, FIG. 14 (represented by reference numeral 116 in FIG. 14) but the probe system is replaced, facilitating significant miniaturisation.

FIGS. 19a and 19b show a simplified schematic illustration of the components of an integrated probe as provided in place of the probe system shown in FIGS. 14 to 16.

The integrated probe system comprises a slit plate 146 provided with a slit 148 and a plurality of illumination sources 151 provided thereon. Preferably, the illumination sources 151 are VCSELs although other integrated illumination sources can be used. Typically, the slit size will be dictated by any or all of requirements for spectral resolution, throughput and spectrometer complexity. Typically though, slit dimensions might be between 10 and 200 micrometres wide and between 800 and 1600 micrometres long. Thus, the size and scale of the slit in comparison to the probe system 114 shown in FIG. 15 is significantly smaller.

FIG. 19b shows a side view of the system of FIG. 19a. As can be seen, the plural VCSELs 151 are arranged, in this example, on the slit plate 146. The VCSELs are arranged in two longitudinal arrays extending along the longitudinal edges of the slit. Individual or group control of the VCSELs is possible which allows to adjust the excitation power. The overall arrangement of the probe is substantially planar such that the width of the probe in its entirety (represented schematically by the dimension “X”) is between 0.5 and 10 mm, and preferably between 1 and 5 mm or more preferably between 1 and 3 mm.

FIG. 20 is a schematic view of the control system used to control the integrated probe of the present application.

The control system includes a controller 152 which is typically a microprocessor or an ASIC. The controller 152 is coupled to a laser driver/controller 154 which itself is coupled to the VCSELs 156. A power supply 158 is provided to provide operating power to the laser driver/controller. Typically, the components illustrated schematically in FIG. 20 are all integrated onto a single unitary PCB such as the slit plate 146 shown in FIG. 19a. The specific configuration of optical sources shown in FIG. 19a, i.e. a single row of optical sources 151 provided on each longitudinal side of the slit, is not a limitation of this configuration. Indeed, the general schematic control system shown in FIG. 20 can be provided with different arrangements of optical sources to that shown in FIG. 19A. Indeed any actual orientation or arrangement of VCSELs 150 on the slit plate 146 can be provided.

The components of the control system, as shown schematically in FIG. 20, can be provided on the same PCB as that on which the VCSELs or optical sources are arranged. In an alternative, they are provided within the envelope or housing of the spectrometer, e.g. within the spectrometer 150 as shown in FIG. 18. In any event, the provision of these components does not detract from the generally planar nature of the probe 148.

FIG. 21 shows an exploded side view of the integrated probe shown more schematically in FIG. 19b. In the example, the slit plate or PCB 146 is provided with the illumination sources 151, typically VCSELs, provided thereon. Illumination source optics 160 is provided. Typically, this could be in the form of micro lenses or an optical window. In addition, slit optics are provided such as a lens which, in one embodiment, could be combined with the illumination source optics 160. As mentioned above with reference to FIG. 19B, the overall arrangement of the probe is substantially planar such that the width of the probe in its entirety (represented schematically by the dimension “X”) is between 0.5 and 10 mm, and preferably between 1 and 5 mm or 1 and 3 mm.

An optical filter 164 is provided in the form of a Rayleigh filter. In this example, the Rayleigh filter 164 is provided behind the slit plate 146, but it will be appreciated that it can be provided on the other of the slit plate 146 as well. It will be appreciated that the probe assembly 148 is effectively planar which means that it can be provided in position on the side of the spectrometer, e.g., the spectrometer 150 in FIG. 18. The overall footprint of the system including the probe 148 and the spectrometer 150 is, in effect, substantially the same as that of the spectrometer 150 alone.

Referring again to FIGS. 19 and 21, optionally, a metal slit plate is provided. The metal slit plate is provided to provide a control over the dimensions of the slit in the PCB or slit plate 146. For example, in situations where the slit specifications or tolerances cannot be met by PCB tooling, a metal plate slit can be attached to the system. The metal plate slit can be placed both on the top side or the back side of the PCB. In the example shown, it is provided on the back side of the PCB. The metal slit plate is denoted by reference numeral 166. As will be appreciated, the integrated probe combines the functionality of the various components shown in and described with reference to FIG. 14 in such a way that significant miniaturisation can be achieved. In practice, the entire volume of the probe such as that shown in FIG. 17 and indicated by reference numeral 114, can be replaced by a single integrated PCB.

FIG. 22 shows a schematic representation of an exemplary set up for the optics. In this example, a 34 by 3 array of single-mode VCSEL emitters 151 is provided at a pitch of 28 micrometres provided on a wafer 168. The arrangement represents a wafer-engineered slit-optic set up. The example minimises the number of additional components required by directly engineering a window 174 into the VCSEL wafer 168. The window 174 is coated with a long-pass filter is directly engineered into the VCSEL wafer 168 using a transparent material. This allows small displacement between the emitter arrays and the detection optical axis 173. Typically, the displacement between the emitter arrays and the detection optical axis 173 will be less than 100 micrometres.

Reformatting optics 170 can be provided. The reformatting optics is in the form a lens and is used to maximise the throughput and magnify the scattered distribution onto the existing slit 172. Typically, this boosts throughput by at least 10%. Precision alignment will be required between the assembly and the existing slit of the spectrometer.

The VCSEL wafer 168 including the transparent window 174 is shown as a merely exemplary configuration for the integrated probe.

It is possible that the VCSEL wafer 168 is provided in two sections, one on either longitudinal side of the transparent window 174 and they are then machined or connected together with the transparent window 174 in a known manner.

The spectrometer slit is typically dimensioned such as to have a width of 100 micrometres and a length of 1300 micrometres and a numerical aperture of 0.22.

The optical flux of each emitter in this example is typically 1.5 mW representing a total optical flux of 306 mW. Preferably the wavelength is between 760 and 850 nanometres although VCSELs of any desired wavelength can be chosen for use in the system. The indication of wavelength ranges given above applies equally here.

FIG. 23 shows a further example of an integrated probe. In this example, angled illumination optics (“direction optics”) 176 are provided on each side of the PCBs 168 including the VCSELs. Preferably, a micro lens array 178 is provided between the PCBs 168 and the direction optics 176. Each emitter array is now separated from the central detection axis by at least 1 mm. This allows the VCSEL arrays to be mounted in separate packages either side of the slit. The micro lens arrays are placed on top of the VCSEL arrays to collimate the light output. This is required due to the increased distance between the sources and the tissue. The direction optics 176 tilt the illumination at a large angle. Again, reformatting optics 180 are provided to boost the throughput. This configuration relies on precise placement of the VCSEL packages with respect to the slit and reformatting optics 180.

FIGS. 24a and 24b show plan and cross-sectional views of an exemplary system similar to that described above with reference to any of FIGS. 19 to 23. In this example, a PCB 182 is provided having VCSELs 184 arranged thereon.

The VCSEL packages 184 are arranged on either side of the slit. In this example, the slit is typically 1500 micrometres long and 100 micrometres wide.

As can be seen, the PCB is arranged on a backing plate 186 and aligned with an opening 190 therein.

FIG. 25 shows a cross sectional view of an exemplary probe. The probe is shown arranged in contact with a sample 192. The sample could be a user's skin if being used for in vivo determination of an analyte concentration or it could be any other sample under analysis as explained above. The probe includes an optical element in the form of a generally transparent structure. The optical element has a generally central region 194 with truncated triangular cut-out 196. The cut-out 196 has sloped edges 193 with a light blocking coating and a generally flat upper surface 191 parallel to the upper surface 197 of the optical element.

The generally flat upper surface is coated with a long pass filter and represents the spectrometer entrance slit. A VCSEL block or die 198 is provided on each side of the slit. Each VCSEL die 198 includes a VCSEL array with possible micro optics 210 arranged on top of them. Transmitted light from the VCSEL array passes through inlet surface 200 on an underside of an overhang within the optical element 195. Angled surfaces 202 totally internally reflect the light for onward transmission to the sample 192. Semiconductor pads 204 and 206 are arranged on the support surface 208. The VCSELS will be operated by control circuitry (not shown) which will be connected to the pads. The arrangement of FIG. 25 thus provides a compact and simple probe arrangement in which the probe is generally planar

Embodiments of the present invention have been described with particular reference to the examples illustrated. However, it will be appreciated that variations and modifications may be made to the examples described within the scope of the present invention.

1. Apparatus for non-invasive in vivo measurement by Raman spectroscopy of analyte presence and/or concentration, such as glucose, in the skin of a subject, the apparatus comprising;
at least one detector;
one or more vertical-cavity surface-emitting lasers spatially separated from the at least one detector, for irradiating the skin of a subject;
wherein the at least one detector is configured to receive Raman scattered radiation transmitted from the sample in response to the received radiation from the one or more vertical-cavity surface-emitting lasers.

2. Apparatus according to claim 1, comprising a plurality of vertical-cavity surface-emitting lasers separated from the at least one detector.

3. Apparatus according to claim 2, wherein the vertical-cavity surface-emitting lasers are arranged as at least one ring of vertical-cavity surface-emitting lasers.

4. Apparatus according to claim 2 or 3, wherein the at least one detector is surrounded by a plurality of rings of vertical-cavity surface-emitting lasers.

5. Apparatus according to any of claims 1 to 4, comprising a plurality of detectors surrounded by a common ring of vertical-cavity surface-emitting lasers.

6. Apparatus according to any of claims 2 to 5, comprising a plurality of detectors surrounded by shared rings of vertical-cavity surface-emitting lasers.

7. Apparatus according to any of claims 2 to 6, wherein the vertical-cavity surface-emitting lasers are configured to provide at least two different wavelengths of radiation to irradiate a sample.

8. Apparatus according to any of claims 2 to 7, wherein the vertical-cavity surface-emitting lasers are configured to provide at collimated beams of light to irradiate the subject, the beams from two or more of the vertical-cavity surface-emitting lasers being arranged to intersect at a defined position.

9. Apparatus for non-invasive in vivo measurement, by Raman spectroscopy, of analyte presence and/or concentration, such as glucose, in the skin of a subject, the apparatus comprising;
at least one radiation source, for irradiating a sample in the skin of a subject; and
a plurality of detectors spatially distributed around the radiation source, wherein the plurality of detectors are configured to receive Raman scattered radiation from the sample in response to the received radiation from the at least one radiation source.

10. Apparatus according to claim 9, wherein the at least one radiation source is a vertical-cavity surface-emitting laser.

11. Apparatus according to claim 9 or 10, wherein the at least one radiation source is surrounded by at least one ring of detectors.

12. Apparatus according to claim 9 or 10, wherein the at least one radiation source is surrounded by a plurality of rings of detectors.

13. Apparatus according to claim 9 or 10, comprising a plurality of radiation sources surrounded by at least one ring of detectors.

14. Apparatus according to claim 9 or 10, comprising a plurality of radiation sources surrounded by a shared plurality of rings of detectors.

15. Apparatus according to claim 13 or 14, wherein the radiation sources are configured to provide at least two different wavelengths of radiation to irradiate a sample.

16. Apparatus according to any of the previous claims, wherein the apparatus further comprises an analysis unit configured to analyse the detected Raman spectrum and infer glucose levels within a sample.

17. Apparatus according to claim 16, wherein the analysis unit is further configured to eliminate background radiation and highlight the Raman spectrum of a sample.

18. Apparatus according to any of the previous claims, comprising a focusing device for focusing the spectrum of Raman scattered radiation transmitted back from the sample for detection.

19. Apparatus according claim 18, wherein the focusing device comprises of at least one optical lens.

20. Apparatus according to claim 19, wherein the at least one optical lens is a convex lens.

21. Apparatus according to claim 18, wherein the focusing device comprises a plurality of optical lenses.

22. Apparatus according to claim 21, wherein plurality of optical lenses comprises a plurality of convex and/or concave lenses.

23. Apparatus according to any of claims 18 to 22, wherein the focusing device comprises one or more components selected from the group including a mirror, an optical fibre, a bundle of optical fibres.

24. A method for non-invasive in vivo measurement, by Raman spectroscopy, of analyte presence and/or concentration, such as glucose, in the skin of a subject, the method comprising;
using the apparatus of any of the previous claims to detect and measure the spectrum of Raman scattered radiation from a sample in the skin of a subject; and
analysing the spectrum of the detected Raman scattered radiation to determine the presence and/or concentration of analyte in the skin of a subject.

25. A method according to claim 24, comprising controlling the vertical-cavity surface-emitting lasers to vary collection depth of the Raman scattered radiation.

26. A method according to claim 25, comprising executing an algorithm to determine the Raman spectrum in dependence on the respective positions of the at least one radiation source and at least one detector relative to the position of the sample.

27. A method according to claim 26, executing the algorithm to eliminate background fluorescence.

28. A method according to claim 27, in which the algorithm utilizes Shift-Excitation Raman Difference Spectroscopy.

29. A method according to claim 27 or 28, comprising:
eliminating non-Raman background fluorescence by comparing the shifts in spectral peaks of observed scattered radiation from a sample, irradiated by at least two different wavelengths;
removing spectral features that do not shift between the spectra created by the at least two difference wavelengths of radiation; and
analysing remaining spectral peaks, for the presence of analyte within the sample.

30. Apparatus for non-invasive in vivo measurement, by Raman spectroscopy, of analyte presence and/or concentration, such as glucose, in the skin of a subject, the apparatus comprising;
at least one detector;
a controllable VCSEL radiation source spaced from the at least one detector, for irradiating the skin of a subject with light, and being configured to selectively change the wavelength of the light in accordance with a SWEPT methodology;
a bandpass filter to receive Raman scattered radiation transmitted back from the sample;
a processor to generate a Raman spectrum from the received Raman scattered radiation.

31. Apparatus for non-invasive in vivo measurement, by Raman spectroscopy, of analyte presence and/or concentration, such as glucose, in the skin of a subject, the apparatus comprising;
at least one detector
a radiation source for irradiating the skin of a subject, spaced from the at least one detector;
wherein the at least one detector is configured to receive a spectrum of Raman scattered radiation transmitted back from the sample in response to the received radiation from the radiation source.

32. Apparatus for non-invasive in vivo measurement by Raman spectroscopy of analyte presence and/or concentration, such as glucose, in the skin of a subject, the apparatus comprising;
a spectrometer having a slit for receiving a Raman spectrum from a sample;
an integrated probe for coupling to the spectrometer, wherein the probe is of generally planar configuration.

33. Apparatus according to claim 32, in which the integrated probe comprises a PCB having arranged thereon plural optical sources and arranged around the slit of the spectrometer.

34. Apparatus according to claim 33, in which the plural optical sources are VCSELs.

35. Apparatus according to claim 33 or 34, in which the PCB comprises a window and the optical sources are arranged around the window.

36. Apparatus according to claim 35, in which plural rows of VCSELs are provided on either side of the window.

37. Apparatus according to any of claims 32 to 36, comprising directing optics to control the distance of the focal point of the optical sources from the plane of the planar integrated probe.

38. Apparatus according to any of claims 32 to 37, comprising source optics arranged for controlling the transmission of light from the optical sources.

39. Apparatus according to claim 38, in which the source optics comprises a micro lens array.

40. Apparatus according to any of claims 32 to 39, wherein the apparatus is for non-invasive in vivo measurement by Raman spectroscopy

41. An integrated probe for coupling to a spectrometer, for non-invasive in vivo measurement by Raman spectroscopy of analyte presence and/or concentration, such as glucose, in the skin of a subject, wherein the probe is of generally planar configuration.

42. An integrated probe according to claim 41, in which the probe is for non-invasive in vivo measurement by Raman spectroscopy of analyte presence and/or concentration.

43. Apparatus for Raman spectroscopy, the apparatus comprising;
a spectrometer having a slit for receiving a Raman spectrum from a sample under test;
an integrated probe for coupling to the spectrometer, wherein the probe is of generally planar configuration.
